(19) United States
(12) Patent Application Publication
(10) Pub. No.: US 2024/0382460 A1
(43) Pub. Date: Nov. 21, 2024

(54) Compositions and Methods for Preventing or Improving Premature Skin Aging

(71) Applicant: NANJING NUTRABUILDING BIO-TECH CO., LTD., Nanjing (CN)

(72) Inventors: Rui SHU, Nanjing (CN); Mingru WANG, Nanjing (CN); Shawn WELLS, Nanjing (CN); Kylin LIAO, Nanjing (CN)

(21) Appl. No.: 18/785,602

(22) Filed: Jul. 26, 2024

Related U.S. Application Data
(63) Continuation-in-part of application No. PCT/CN2023/072356, filed on Jan. 16, 2023.

(30) Foreign Application Priority Data
Jan. 28, 2022 (WO) PCT/CN2022/074500

(51) Int. Cl. A61K 31/4172; A61K 8/49; A61K 45/06; A61P 17/18; A61Q 19/08

(52) U.S. Cl. CPC A61K 31/4172; A61K 8/4946; A61K 45/06; A61P 17/18; A61Q 19/08

(57) ABSTRACT

The invention provides a topical composition for preventing or treating skin aging, comprising at least one compound selected from the group consisting of ergothioneine, an analog or derivative thereof, or a dermatologically acceptable salt thereof. This topical composition improves skin's radiance, luminosity or texture, and rarely produces skin irritation. In another aspect, this invention provides a method for preventing or treating photoaging in a subject, comprising topical application of the composition of the invention.

### CROSS REFERENCE TO RELATED APPLICATION

This application is a continuation-in-part application of International Patent Application No. PCT/CN2023/072356, filed on Jan. 16, 2023, which claims the priority of the international application No. PCT/CN2022/074500, filed on Jan. 28, 2022, the contents of all of which are incorporated herein by reference in their entirety.

### FIELD OF THE INVENTION

One aspect of the invention relates to a topical composition, comprising at least one ergothioneine, an analog or derivative thereof, or a dermatologically acceptable salt thereof, for preventing or treating skin aging, particularly, premature aging. Another aspect of the invention relates to a method for treating skin aging, comprising topical application of the composition of the invention. This topical composition has been reported to rarely produce skin irritation.

### BACKGROUND OF THE INVENTION

Skin is the main barrier that segregate our body from the outer environment and protects us against environmental stressors. However, like other human organs, skin is also subject to the aging process. Skin aging is a complex biological phenomenon, influenced by both intrinsic factors (e.g., genetics, cellular metabolism, hormone environment) and extrinsic factors (e.g., chemicals, toxins, pollutants, ultraviolet (UV), and ionizing radiation), and affecting different constituents of the skin. Intrinsic aging is an inevitable, genetically determined process that affects unexposed and exposed skin and is characterized by dryness, laxity, fine wrinkles, and skin atrophy, while extrinsic aging is engendered by external environment factors such as air pollution, smoking, poor nutrition, and sun exposure, resulting in coarse wrinkles, loss of elasticity, laxity, and rough-textured appearance (Cell Transplant. 2018 May; 27 (5): 729-738). While many external factors could contribute to skin aging, ultraviolet (UV) radiation from sunlight is the primary factor of extrinsic skin aging, also referred to as premature skin aging or photoaging. UV irradiation can induce non-genomic signal transduction. Generally speaking, UV irradiation induce generation of reactive oxygen species (ROS). When UV-induced ROS exceeds the capacity of cells to chemically reduce ROS, then the consequence is oxidative stress. ROS play an important role in UV-induced signal transduction and gene expression, reflecting central aspects of skin aging (Journal of Dermatological Science Supplement Volume 1, Issue 2, December 2005, Pages S1-S8, https://doi.org/10.1016/j.descs.2005.06.002).

Unlike intrinsic aging, photoaging can be controlled to an extent as it is a result of external factors. Photoaging affects the sun-exposed areas and is characterized clinically by fine and coarse wrinkling, roughness, dryness, laxity, and pigmentary changes. There is also an increase in development of benign and malignant neoplasms on photoaged skin (Journal of the American Academy of Dermatology, Volume 49, Issue 4, October 2003, Pages 690-697). The anti-aging strategies to combat skin aging signs and dysfunction have been developed over the last decades. As people's cosmetic requirements increase, more research efforts are conducted to help relieving skin aging or preventing of extrinsic aging from occurring.

Ergothioneine (EGT) is a naturally occurring, sulfur-containing amino acid, and is presumed to function as a natural antioxidant. It is mainly found in mushrooms, but also in king crab, and animals that have grazed on grasses containing ergothioneine. Since humans or other vertebrates are unable to synthesize ergothioneine, it can only be acquired by diet. Studies in animals and humans have found no toxicity or adverse effects to be associated with ergothioneine administration, even at high doses. Recently, ergothioneine (Tetrahedron, Paris, France) has attained European Food Safety Authority approval as a Novel Food in the European Union and is generally recognized as a safe supplement and food ingredient by the Food and Drug Administration in the US (GRAS notice No. 734) (FEBS Letters 592 (2018) 3357-3366).

Applicants of the present invention have investigated the safety and efficacy of a composition comprising ergothioneine on alleviating skin aging, and provided a topical composition comprising at least one ergothioneine, an analog or derivative thereof, or a dermatologically acceptable salt thereof, which can counteract or delay skin aging signs in a safe and effective way. This topical composition has been reported to rarely produce skin irritation.

### SUMMARY OF THE INVENTION

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

This invention generally relates to compositions for preventing or improving skin aging, containing at least an active compound which is ergothioneine, an analog, derivative, or dermatologically acceptable salt thereof. Additionally, this invention also relates to a topical application of the composition of the invention to an area of the skin for preventing or treating of damages to skin, particularly, skin damages resulting from premature aging (also known as photoaging).

One aspect of the present invention provides a composition for preventing or treating skin aging, comprising at least one compound selected from the group consisting of ergothioneine, an analog or derivative thereof, or a dermatologically acceptable salt thereof.

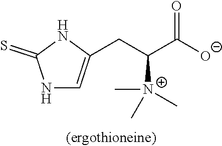

In some embodiments, ergothioneine, an analog or derivative thereof, or a dermatologically acceptable salt thereof is present in an amount ranging from 0.01 to 20% by weight (w/w), 0.01 to 15% by weight (w/w), or 0.01 to 12% by weight (w/w).

In some embodiments, ergothioneine, an analog or derivative thereof, or a dermatologically acceptable salt thereof, is present in an amount of 10% by weight (w/w). In some other embodiments, ergothioneine, an analog or derivative thereof, or a dermatologically acceptable salt thereof, is present in an amount of 0.5% by weight (w/w).

In some embodiments, the analog or derivative of ergothioneine is selected from the group consisting of S-Methyl-L-Ergothioneine, N-Desmethyl L-Ergothioneine Methyl Ester, N(Im)-Ethoxycarbonyl-S-ethoxycarbonyl L-Ergothioneine Methyl Ester lodide, N-Desmethyl L-Ergothioneine-D6 Methyl Ester, L-(+)-Ergothioneine-D3, ergothioneine sulfonate, and L-Ergothioneine Hydrochloride.

In some embodiments, ergothioneine is L-ergothioneine.

In some embodiments, the composition further comprises at least one additional agent. Examples of the additional agent include, but not limited to, growth factor, Hyaluronic Acid, Retinol/Retinoic acid, Hydrolyzed Collagen/Collagen Peptides, Vitamin C, Vitamin E, Cannabidiol, EGCG/Green tea, Polyhydroxy Acids, Glycolic Acid, Aloe vera, Niacinamide, Resveratrol, Superoxide dismutase, Tea tree oil, Bakuchiol, Grapeseed oil, Petroleum jelly, and squalene. Examples for growth factors are, but not limited to, EGF, FGF, NGF, PDGF, VEGF, IGF, GMCSF, GCSF, TGF, Erythropieitn, TPO, BMP, HGF, GDF, Neurotrophins, MSF, SGF, GDF.

In some embodiments, the skin aging comprises photoaging.

In some embodiments, preventing or improving photoaging is achieved by protection against UV-induced mtDNA deletion. In some embodiments, preventing or improving skin aging can be achieved by reducing or alleviating mtDNA deletion.

Example of photoaging includes, but not limited to, wrinkling, scar tissue deposition, altered skin elasticity, altered skin color, altered skin texture, altered skin thickness, angioma, telangiectasia, sunburn, dryness, itchiness, neoplasia and precancerous growth.

In some embodiments, the preventing or improving treating skin aging comprises one or more of the following effects: alleviating skin photoaging, diminishing skin wrinkles, improving skin elasticity, improving skin firmness, or improving skin's radiance, luminosity, or texture. In some particular embodiments, the composition improves skin's radiance, luminosity, or texture, and rarely produces skin irritation.

In some embodiments, the composition is prepared in any one formulation selected from the group consisting of a skin lotion, a skin softener, a skin toner, a skin cream, a sunscreen, an essence, a mask pack, a mask sheet, a soap, a shampoo, a cleaning foam, a cleaning lotion, a cleaning cream, an ointment, a gel and a massage cream.

Another aspect of the invention relates to a method for preventing or improving skin aging in a subject, comprising topically applying the composition, comprising at least one compound selected from the group consisting of ergothioneine, an analog or derivative thereof, or a dermatologically acceptable salt thereof, to an area of the skin.

In some embodiments, ergothioneine, an analog or derivative thereof, or a dermatologically acceptable salt thereof, is present in an amount ranging from 0.01 to 20% by weight (w/w), 0.01 to 15% by weight (w/w), or 0.01 to 12% by weight (w/w).

In some embodiments, the subject is human.

In some embodiments, ergothioneine is L-ergothioneine. In some embodiments, the skin aging comprises photoaging.

In some embodiments, preventing or improving photoaging is achieved by protection against UV-induced mtDNA deletion. In some embodiments, preventing or improving skin aging can be achieved by reducing or alleviating mtDNA deletion.

In some embodiments, the preventing or improving treating skin aging comprises one or more of the following effects: alleviating skin photoaging, diminishing skin wrinkles, improving skin elasticity, improving skin firmness, or improving skin's radiance, luminosity, or texture. In some particular embodiments, the preventing or improving treating skin aging comprises one or more of the following effects: improving skin's radiance, luminosity, or texture, and rarely produces skin irritation.

A further aspect of the invention relates to use of ergothioneine in manufacturing a composition capable of treating skin photoaging.

### BRIEF DESCRIPTION OF THE FIGURES

FIG. 1 depicts the change of Methylobacterium-Methylorubrum after 12-week's treatment of Ergothioneine.

FIG. 2 depicts the effect of compound ergothioneine on the UV-induced mtDNA common deletion (Cdel) in fibroblasts.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. To the contrary, the invention is intended to cover alternatives, modifications and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

Generally speaking, various embodiments of the present invention provide for compositions, comprising at least one compound selected from the group consisting of ergothioneine, an analog or derivative thereof, or a dermatologically acceptable salt thereof; as well as methods for preventing or treating skin aging, comprising topical application of the composition of the invention to an area of skin. Moreover, the composition can be prepared in a variety of forms, such as a skin lotion, a skin softener, a skin toner, a skin cream, a sunscreen, an essence, a mask pack, a mask sheet, a soap, a shampoo, a cleaning foam, a cleaning lotion, a cleaning cream, an ointment, a gel, and a massage cream.

### Definitions

As used herein, the term “or” is meant to include both “and” and “or”. In other words, the term “or” may also be replaced with “and/or.”

As used herein, the singular forms “a,” “an” and “the” are intended to include the plural forms as well, unless the context clearly indicates otherwise.

As used herein, the term “at least” followed by a number is used to denote the start of a range beginning with that number.

As used herein, the term “comprise” or “include” and their conjugations, refer to a situation where said terms are used in their non-limiting sense to mean that items following the word are included, but items not specifically mentioned are not excluded. It also encompasses the more limiting verb ‘to consist essentially of’ and ‘to consist of’.

As used herein, the term “subject” or “patient” is used interchangeably and as used herein mean any mammal including but not limited to human beings including a human patient or subject to which the compositions of the invention can be administered. The term “mammals” include human patients and non-human primates, as well as experimental animals such as rabbits, rats, and mice, and other animals.

As used herein, the term “treat”, “treating” or “treatment” and the like as used herein, refers to any indicia of success in the prevention or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluations. Accordingly, the term “treating” or “treatment” includes the administration of L-Ergothioneine which may be in combination with other compounds or agents.

As used herein, the terms “improving” and the like are used generally to mean improving or alleviating it or its symptoms partially in the subject, such as alleviating skin photoaging, diminishing skin wrinkles, improving skin elasticity, improving skin firmness, or improving skin's radiance, luminosity, or texture.

As used herein, the terms “preventing,” “prevention,” and the like are used generally to mean preventing or inhibiting deterioration or further deterioration of the visual system of an aging subject, as compared with a comparable visual system not receiving the synthetic retinal derivative.

As used herein, the term “effective amount” refers to an amount that is required to improve at least one symptom of a medical condition in an individual.

Compounds and compositions described herein can be employed for cosmetic or dermatological uses. A “cosmetic”, as used herein, can be contacted with the skin, such as by being rubbed, poured, sprinkled, or sprayed on the skin or otherwise introduced into or onto the skin, and is intended to improve the aesthetic appearance of the skin, such as by cleaning, beautifying, promoting attractiveness, or altering, e.g., improving, the aesthetic appearance of the contacted skin. A cosmetic benefit is typically visual or aesthetic, and can be evaluated using subjective or objective assays.

As used herein, the term “topical application”, “topically applying” and the like, means directly laying on or spreading on the skin, hair, or nail, e.g., by use of the hands or an applicator such as a wipe.

As used herein, the term “dermatologically acceptable” is used to refer to compounds and compositions that retain biological effectiveness and which are suitable for use in contact with the skin or hair of humans and lower animals without undue toxicity, incompatibility, instability, irritation, allergic response, and the like.

### Method 1 Clinical Efficacy Study

Thirty healthy female subjects with mild to moderate photoaging and Fitzpatrick skin types I-II and 35-70 years of age are enrolled for a clinical efficacy study. Inclusion criteria and exclusion criteria are considered.

Following enrollment, subjects will be randomly assigned to use 0.01-12% ergothioneine moisturizer of the invention to the right or left face and the bland moisturizer to the opposite face. Neither the subjects nor the investigator will know the identity of the creams. The study coordinator will develop and maintain the study blind. Subjects will be dispensed a diary to record daily use of the moisturizers, provided a return schedule, and asked to return to the research center at week 4, 8 and 12. On each return (check point), the enrolled subjects will complete baseline efficacy and tolerability assessments separately for each side of the face. The skin elasticity and corneometry measurements will be made and recorded from both sides of the face. Additionally, at week 8 and 12, ten randomly selected subjects will participate in the photography substudy. VISIA-CR images will be taken of the front, right, and left face with standard lighting 1 at baseline and week 12 in both jpeg and CR2 raw files for image analysis at a later date. Furthermore, at week 12 (the study endpoint), both sides of the face are swabbed for microbiome analysis. Subject diaries will be checked for compliance and collected.

### Efficacy Assessment

The following assessments will be made for data collection purposes:

Dermatologist Investigator assessed efficacy parameters: fine lines, wrinkles, texture, radiance, luminosity, smoothness, skin tone evenness, firmness, and pores. All assessments will be made on a 5-point ordinal scale (0=none, 1=minimal, 2=mild, 3=moderate, 4=severe) at baseline, week 4, week 8, and week 12. Each side of the face will be assessed separately (Data will be recorded in the Table below).

| Visits | Baseline | Week 4 | Week 8 | Week 12 |
|---|---|---|---|---|
| Fine Lines | 0 . . . 1 . . . | 0 . . . 1 . . . | 0 . . . 1 . . . | 0 . . . 1 . . . |
| Score | 2 . . . 3 . . . 4 | 2 . . . 3 . . . 4 | 2 . . . 3 . . . 4 | 2 . . . 3 . . . 4 |
| Wrinkles | 0 . . . 1 . . . | 0 . . . 1 . . . | 0 . . . 1 . . . | 0 . . . 1 . . . |
| Score | 2 . . . 3 . . . 4 | 2 . . . 3 . . . 4 | 2 . . . 3 . . . 4 | 2 . . . 3 . . . 4 |
| Poor Skin | 0 . . . 1 . . . | 0 . . . 1 . . . | 0 . . . 1 . . . | 0 . . . 1 . . . |
| Texture | 2 . . . 3 . . . 4 | 2 . . . 3 . . . 4 | 2 . . . 3 . . . 4 | 2 . . . 3 . . . 4 |
| Score | | | | |
| Radiance | 0 . . . 1 . . . | 0 . . . 1 . . . | 0 . . . 1 . . . | 0 . . . 1 . . . |
| Score | 2 . . . 3 . . . 4 | 2 . . . 3 . . . 4 | 2 . . . 3 . . . 4 | 2 . . . 3 . . . 4 |
| Luminosity | 0 . . . 1 . . . | 0 . . . 1 . . . | 0 . . . 1 . . . | 0 . . . 1 . . . |
| Score | 2 . . . 3 . . . 4 | 2 . . . 3 . . . 4 | 2 . . . 3 . . . 4 | 2 . . . 3 . . . 4 |
| Smoothness | 0 . . . 1 . . . | 0 . . . 1 . . . | 0 . . . 1 . . . | 0 . . . 1 . . . |
| Score | 2 . . . 3 . . . 4 | 2 . . . 3 . . . 4 | 2 . . . 3 . . . 4 | 2 . . . 3 . . . 4 |
| Skin Tone | 0 . . . 1 . . . | 0 . . . 1 . . . | 0 . . . 1 . . . | 0 . . . 1 . . . |
| Evenness | 2 . . . 3 . . . 4 | 2 . . . 3 . . . 4 | 2 . . . 3 . . . 4 | 2 . . . 3 . . . 4 |
| Score | | | | |
| Firmness | 0 . . . 1 . . . | 0 . . . 1 . . . | 0 . . . 1 . . . | 0 . . . 1 . . . |
| Score | 2 . . . 3 . . . 4 | 2 . . . 3 . . . 4 | 2 . . . 3 . . . 4 | 2 . . . 3 . . . 4 |
| Pores | 0 . . . 1 . . . | 0 . . . 1 . . . | 0 . . . 1 . . . | 0 . . . 1 . . . |
| Score | 2 . . . 3 . . . 4 | 2 . . . 3 . . . 4 | 2 . . . 3 . . . 4 | 2 . . . 3 . . . 4 |

Dermatologist Investigator assessed tolerability parameters: peeling, dryness, redness, and swelling. All assessments will be made on a 5-point ordinal scale (0=none, 1=minimal, 2=mild, 3=moderate, 4=severe) at baseline, week 4, week 8, and week 12. Each side of the face will be assessed separately (Data will be recorded in the Table below).

| Visits | Baseline | Week 4 | Week 8 | Week 12 |
|---|---|---|---|---|
| Peeling | 0 . . . 1 . . . | 0 . . . 1 . . . | 0 . . . 1 . . . | 0 . . . 1 . . . |
| | 2 . . . 3 . . . 4 | 2 . . . 3 . . . 4 | 2 . . . 3 . . . 4 | 2 . . . 3 . . . 4 |
| Dryness | 0 . . . 1 . . . | 0 . . . 1 . . . | 0 . . . 1 . . . | 0 . . . 1 . . . |
| | 2 . . . 3 . . . 4 | 2 . . . 3 . . . 4 | 2 . . . 3 . . . 4 | 2 . . . 3 . . . 4 |
| Redness | 0 . . . 1 . . . | 0 . . . 1 . . . | 0 . . . 1 . . . | 0 . . . 1 . . . |
| | 2 . . . 3 . . . 4 | 2 . . . 3 . . . 4 | 2 . . . 3 . . . 4 | 2 . . . 3 . . . 4 |
| Swelling | 0 . . . 1 . . . | 0 . . . 1 . . . | 0 . . . 1 . . . | 0 . . . 1 . . . |
| | 2 . . . 3 . . . 4 | 2 . . . 3 . . . 4 | 2 . . . 3 . . . 4 | 2 . . . 3 . . . 4 |

Subject assessed efficacy parameters: fine lines, wrinkles, texture, radiance, luminosity, smoothness, skin tone evenness, firmness, and pores. All assessments will be made on a 5-point ordinal scale (0=none, 1=minimal, 2=mild, 3=moderate, 4=severe) at baseline, week 4, week 8, and week 12. Each side of the face will be assessed separately (Data will be recorded in the Table below).

| Visits | Baseline | Week 4 | Week 8 | Week 12 |
|---|---|---|---|---|
| Fine Lines | 0 . . . 1 . . . | 0 . . . 1 . . . | 0 . . . 1 . . . | 0 . . . 1 . . . |
| Score | 2 . . . 3 . . . 4 | 2 . . . 3 . . . 4 | 2 . . . 3 . . . 4 | 2 . . . 3 . . . 4 |
| Wrinkles | 0 . . . 1 . . . | 0 . . . 1 . . . | 0 . . . 1 . . . | 0 . . . 1 . . . |
| Score | 2 . . . 3 . . . 4 | 2 . . . 3 . . . 4 | 2 . . . 3 . . . 4 | 2 . . . 3 . . . 4 |
| Poor Skin | 0 . . . 1 . . . | 0 . . . 1 . . . | 0 . . . 1 . . . | 0 . . . 1 . . . |
| Texture | 2 . . . 3 . . . 4 | 2 . . . 3 . . . 4 | 2 . . . 3 . . . 4 | 2 . . . 3 . . . 4 |
| Score | | | | |
| Radiance | 0 . . . 1 . . . | 0 . . . 1 . . . | 0 . . . 1 . . . | 0 . . . 1 . . . |
| Score | 2 . . . 3 . . . 4 | 2 . . . 3 . . . 4 | 2 . . . 3 . . . 4 | 2 . . . 3 . . . 4 |
| Luminosity | 0 . . . 1 . . . | 0 . . . 1 . . . | 0 . . . 1 . . . | 0 . . . 1 . . . |
| Score | 2 . . . 3 . . . 4 | 2 . . . 3 . . . 4 | 2 . . . 3 . . . 4 | 2 . . . 3 . . . 4 |
| Smoothness | 0 . . . 1 . . . | 0 . . . 1 . . . | 0 . . . 1 . . . | 0 . . . 1 . . . |
| Score | 2 . . . 3 . . . 4 | 2 . . . 3 . . . 4 | 2 . . . 3 . . . 4 | 2 . . . 3 . . . 4 |
| Skin Tone | 0 . . . 1 . . . | 0 . . . 1 . . . | 0 . . . 1 . . . | 0 . . . 1 . . . |
| Evenness | 2 . . . 3 . . . 4 | 2 . . . 3 . . . 4 | 2 . . . 3 . . . 4 | 2 . . . 3 . . . 4 |
| Score | | | | |
| Firmness | 0 . . . 1 . . . | 0 . . . 1 . . . | 0 . . . 1 . . . | 0 . . . 1 . . . |
| Score | 2 . . . 3 . . . 4 | 2 . . . 3 . . . 4 | 2 . . . 3 . . . 4 | 2 . . . 3 . . . 4 |
| Pores | 0 . . . 1 . . . | 0 . . . 1 . . . | 0 . . . 1 . . . | 0 . . . 1 . . . |
| Score | 2 . . . 3 . . . 4 | 2 . . . 3 . . . 4 | 2 . . . 3 . . . 4 | 2 . . . 3 . . . 4 |

Subject assessed tolerability parameters: itching, stinging, burning, and irritation. All assessments will be made on a 5-point ordinal scale (0=none, 1=minimal, 2=mild, 3=moderate, 4=severe) at baseline, week 4, week 8, and week 12. Each side of the face will be assessed separately (Data will be recorded in the Table below).

| Visits | Baseline | Week 4 | Week 8 | Week 12 |
|---|---|---|---|---|
| Itching | 0 . . . 1 . . . | 0 . . . 1 . . . | 0 . . . 1 . . . | 0 . . . 1 . . . |
| | 2 . . . 3 . . . 4 | 2 . . . 3 . . . 4 | 2 . . . 3 . . . 4 | 2 . . . 3 . . . 4 |
| Stinging | 0 . . . 1 . . . | 0 . . . 1 . . . | 0 . . . 1 . . . | 0 . . . 1 . . . |
| | 2 . . . 3 . . . 4 | 2 . . . 3 . . . 4 | 2 . . . 3 . . . 4 | 2 . . . 3 . . . 4 |
| Burning | 0 . . . 1 . . . | 0 . . . 1 . . . | 0 . . . 1 . . . | 0 . . . 1 . . . |
| | 2 . . . 3 . . . 4 | 2 . . . 3 . . . 4 | 2 . . . 3 . . . 4 | 2 . . . 3 . . . 4 |
| Irritation | 0 . . . 1 . . . | 0 . . . 1 . . . | 0 . . . 1 . . . | 0 . . . 1 . . . |
| | 2 . . . 3 . . . 4 | 2 . . . 3 . . . 4 | 2 . . . 3 . . . 4 | 2 . . . 3 . . . 4 |

Noninvasive assessments: skin elasticity and corneometery hydration measurements will be made and recorded from both the right and left cheek at baseline, week 4, week 8, and week 12 (Data will be recorded in the Table below).

| Visits | Baseline | Week 4 | Week 8 | Week 12 |
|---|---|---|---|---|
| Elasticity | | | | |
| Readings | | | | |
| RIGHT FACE | | | | |
| VE | | | | |
| E | | | | |
| Retraction | | | | |
| LEFT FACE | | | | |
| VE | | | | |
| E | | | | |
| Retraction | | | | |
| Corneometry | | | | |
| Reading | | | | |
| RIGHT FACE | | | | |
| LEFT FACE | | | | |
| Microbiome | | | | |
| Swabbing | | | | |
| RIGHT FACE | | XXXXXXX | XXXXXXX | |
| LEFT FACE | | XXXXXXX | XXXXXXX | |

Photography: 10 subjects will participate in the photography substudy. VISIA-CR images will be taken of the front, right, and left face with standard lighting 1 at baseline and week 12 in both jpeg and CR2 raw files for image analysis at a later date.

Microbiome swabbing: Both sides of the face will be swabbed for microbiome analysis at baseline and week 12. Two swabs will be generated per visit for a total of 4 swabs per subject. Swabs will be stored in the −80° C. freezer until the end of the study for shipping (on dry ice) to a commercial laboratory in Vancouver, Canada for microbiome quantitative profiling. 120 swabs will be shipped at the end of study for analysis.

The following instructions will be followed to obtain the facial swabs:
STEP 1: Dip a fresh swab in molecular grade DNA free water. With one hand, stretch the skin site taut. With the other hand, hold the swab so the shaft is parallel to the skin surface. Apply firm pressure and rub the swab back and forth rigorously 50 times (for 30 seconds) rotating the swab slowly while swabbing.
STEP 2: Carefully return the swab to the tube, ensuring that neither the swab, the swab stick nor the vessel touch any surface.
STEP 3: Place the tube into a biospecimen bag and freeze at −80° C. until ready to ship.

### Method 2 Clinical Safety Study

Although the topical product of the invention has been reported to rarely produce skin irritation, Applicant will conduct a safety study by reporting all adverse experiences (AEs) that occur throughout the study, regardless of severity and whether or not attributed to the product of the invention. The report will include: date of onset, a description of the AE, severity, seriousness, action taken, relationship to the study drug, outcome of the event, and date of resolution.

The intensity or severity of an AE is characterized as:
Mild: AE which is easily tolerated.
Moderate: AE sufficiently discomforting to interfere with daily activity.
Severe: AE which prevents normal daily activities.

All data will be recorded in the Table below.

| | | |
|---|---|---|
| Adverse Event | 1 | 2 |

Start Date (mm/dd/yy)
Stop Date (mm/dd/yy)
Ongoing*
Frequency¹
Severity²
Relation to Study Med³
Action Taken⁴
Outcome⁵
Serious (Y/N)
¹Frequency
²Severity
³Relation to Study Med
⁴Action Taken (insert all codes that apply)
1 = Continuous
1 = Mild
1 = Not related
1 = None
2 = Intermittent
2 = Moderate
2 = Possible
2 = Study drug discontinued
3 = Isolated
3 = Severe
3 = Probable
3 = Non-drug therapy
4 = Definite
4 = New OTC or Rₓ drug added
⁵Outcome
1 = Resolved
2 = Improved
3 = Stabilized
4 = Ongoing
5 = Worsened
6 = Lost to follow-up

The relationship to the study product is characterized as:
Not Related—applies to any adverse experience that is clearly not related to use of the study product.
Possible—means the association of the adverse experience with the study product is unknown; however, a relationship between study product and experience cannot be ruled out.
Probable—there is a reasonable temporal relationship between the use of the study product and the adverse experience. Based upon the investigator's clinical experience, the association of the event with the study product seems likely.
Definite—The AE occurs following the application of the study product and it cannot be reasonably explained by any other known characteristics of the subject's clinical state, environmental or toxic factors, or other modes of therapy administered to the subject. It disappears or decreases upon discontinuation of the study product and reappears on a re-challenge of the study product. This is necessary to evaluate allergic or irritant contact dermatitis.

### Method 3 Statistical Analysis

A Wilcoxon signed rank test is used for analysis of all ordinal nonparametric data as compared to baseline. A Student's T-test is used to analyze all parametric numerical elasticity and corneometry data. An outside lab will analyze the data from the facial swabs. Statistical significance is defined as p value less than or equal to 0.05.

### Method 4 In Vitro Efficacy Study

The normal human dermal fibroblasts (NHDF) were incubated for 16 days, with ultraviolet light exposure three times a day. For the experimental group the cells were treated using medium containing 0.5, 3, 30 μM of ergothioneine.

Deletions in mtDNA were quantified using qPCR. The 522 bp fragment represents the amount of mtDNA with deletions, whereas the 262 bp fragment reflects the quantity of intact mtDNA, Relative intensity (AU) is typically used to describe the intensity of the fluorescent signal, and its magnitude is proportional to the number of fragments measured. And cell activity was analyzed using WST-8.

The following examples are illustrative of select embodiments of the present invention and are not meant to limit the scope of the invention.

### Example 1 Anti-aging Composition for Topical Application

A topical composition comprising ergothioneine was prepared. The amounts of each component of the compositions are given as percentages of each component's weight relative to the total weight of the composition.

The formulation comprises 0.01 to 12% (w/w) ergothioneine, and one or more additives selected from Hyaluronic Acid (0.1-3%), Retinol/Retinoic acid (0.1-2%), Hydrolyzed Collagen/Collagen Peptides (0.1-3%), Vitamin C (0.1-6%), Vitamin E (0.1-10%), Cannabidiol (0.1-5%), EGCG/Green tea (0.1-10%), Polyhydroxy Acids (0.1-12%), Glycolic Acid (0.1-10%), Aloe vera (0.1-10%), Niacinamide (0.1-10%), Resveratrol (0.1-5%), Superoxide dismutase (0.1-5%), Tea tree oil (0.1-10%), Bakuchiol (0.1-5%), Grapeseed oil (0.1-10%), Petroleum jelly (0.1-5%), or squalene (0.1-10%) (w/w).

### Example 2 Clinical Efficacy Study

The dermatologist investigator assessed efficacy of ergothioneine cream at baseline, week 4, week 8, and week 12 in terms of fine lines score, wrinkles score, texture score, radiance score, luminosity score, smoothness score, skin tone evenness score, firmness score, and pores score. All assessments were made on a 5-point ordinal scale (0=none, 1=minimal, 2=mild, 3=moderate, 4=severe). Each side of the face was assessed separately. The summary intergroup analysis is presented in Table 1 below.

TABLE 1

| | Mann-Whitney | Mean | Mean | Active v. |
|---|---|---|---|---|
| | two tailed paired | Active | Control | Control p= |
| Week 4 | Fine Lines Score | 2.54 | 2.57 | 0.791 |
| | Wrinkles Score | 2.54 | 2.57 | 0.791 |
| | Texture Score | 2.54 | 2.61 | 0.595 |
| | Radiance Score | 2.07 | 2.46 | 0.036 |
| | Luminosity Score | 1.75 | 2.18 | 0.026 |
| | Smoothness Score | 1.93 | 2.07 | 0.471 |
| | Tone Evenness Score | 3.00 | 3.00 | 0.835 |
| | Firmness Score | 2.64 | 2.68 | 0.782 |
| | Pores Score | 2.54 | 2.57 | 0.791 |
| Week 8 | Fine Lines Score | 2.54 | 2.54 | 1.000 |
| | Wrinkles Score | 2.54 | 2.54 | 1.000 |
| | Texture Score | 2.21 | 2.57 | 0.044 |
| | Radiance Score | 1.71 | 2.21 | 0.021 |
| | Luminosity Score | 1.50 | 1.96 | 0.017 |
| | Smoothness Score | 1.54 | 1.82 | 0.108 |
| | Tone Evenness Score | 2.82 | 2.96 | 0.480 |
| | Firmness Score | 2.50 | 2.64 | 0.372 |
| | Pores Score | 2.54 | 2.57 | 0.791 |

The investigator assessed overall appearance improvement of the 10% ergothioneine cream treated side of the face as compared to the bland moisturizer treated side after 12 weeks of application. Results showed that there was a statistically significant improvement in radiance (p=0.036) and luminosity (p=0.026) for the active ergothioneine side of the face over the bland moisturizer at week 4. This improvement continued into week 8 for radiance (p=0.021) and luminosity (p=0.017) with the addition of statistically significant improvement in texture (p=0.044) (see Table 1).

In summary, the primary efficacy endpoint was met at week 4 for luminosity and radiance and week 8 for radiance, luminosity, and texture.

The dermatologist investigator assessed peeling, dryness, redness, and swelling. All assessments were made on a 5-point ordinal scale (0=none, 1=minimal, 2=mild, 3=moderate, 4=severe) at baseline, week 4, week 8, and week 12. Each side of the face was assessed separately. The investigator noted no tolerability issues at any time point (see Table 2).

TABLE 2

| | Mann-Whitney | Mean | Mean | Active v. |
|---|---|---|---|---|
| | two tailed paired | Active | Control | Control p= |
| Baseline | Peeling | 0.00 | 0.00 | 1.000 |
| | Dryness | 0.00 | 0.00 | 1.000 |
| | Redness | 0.00 | 0.00 | 1.000 |
| | Swelling | 0.00 | 0.00 | 1.000 |
| Week 4 | Peeling | 0.00 | 0.00 | 1.000 |
| | Dryness | 0.00 | 0.00 | 1.000 |
| | Redness | 0.07 | 0.07 | 1.000 |
| | Swelling | 0.00 | 0.00 | 1.000 |
| Week 8 | Peeling | 0.00 | 0.00 | 1.000 |
| | Dryness | 0.00 | 0.00 | 1.000 |
| | Redness | 0.00 | 0.00 | 1.000 |
| | Swelling | 0.04 | 0.00 | 0.434 |
| Week 12 | Peeling | 0.00 | 0.00 | 1.000 |
| | Dryness | 0.00 | 0.00 | 1.000 |
| | Redness | 0.00 | 0.00 | 1.000 |
| | Swelling | 0.00 | 0.00 | 1.000 |

### Example 3 Clinical Safety Study

The safety endpoint was the absence of significant adverse reactions. The safety endpoint was met.

### Example 4 Microbiome Results

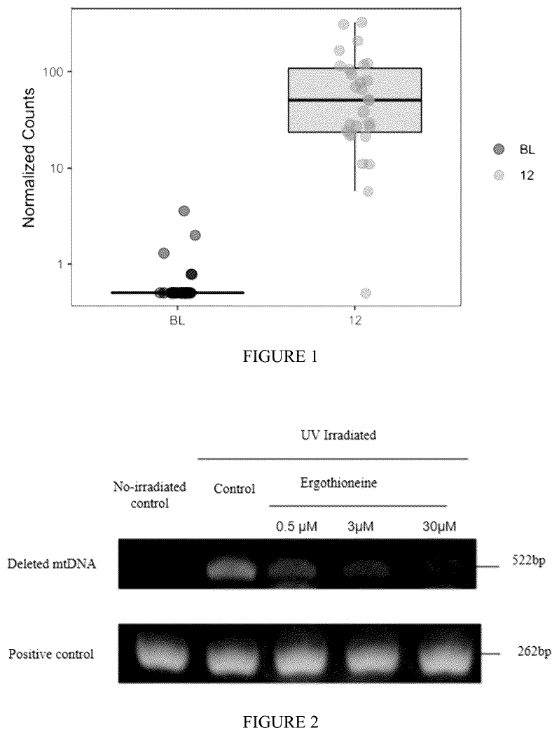

The result is depicted in FIG. 1. The differentially abundant taxa illustrated showed that after 12-week's treatment of Ergothioneine the normalized counts of Methylobacterium-Methylorubrum were significantly increased (P. adj=8.7e-21) from baseline (BL). The results shows that the 10% ergothioneine maintained the existing microbiome profile and increased Methylobacterium-Methylorubrum, which is beneficial to human skin.

### Example 5 In Vitro Efficacy Study

FIG. 2 depicts the effect of compound ergothioneine on the UV-induced mtDNA common deletion (Cdel) in fibroblasts—migration of PCR products (262 bp fragment=total mtDNA without Cdel; 522 bp fragment=mtDNA with Cdel) on 1.5% agarose gel. The results (Table 3 and FIG. 2) demonstrate that UVA irradiation induced a marked increase of the occurrence of common deletion (Cdel) in mtDNA in fibroblasts (60-fold increase compared to the non-irradiated control). Ergothioneine, tested at concentrations of 0.5, 3, and 30 μM, significantly reduced the incidence of Cdel in UV-irradiated NHDF in a concentration-dependent manner. Specifically, compared to the irradiated control group, the reduction in Cdel incidence was 56% at 0.5 μM, 67% at 3 μM, and 75% at 30 μM, suggesting that ergothioneine offers substantial protection against UV-induced mtDNA deletion, thus preventing or improving, or reducing or alleviating skin aging, especially photoaging.

TABLE 3

| | | | | PCR on mtDNA (262 bp) long | | Nest PCR - deleted | | Viability |
|---|---|---|---|---|---|---|---|---|
| | | | | fragment | | mtDNA | | (WST-8) |
| | | | | Relative | % | Relative | % | % |
| | | | | Intensity | Irradiated | Intensity | Irradiated | Irradiated |
| Test compound | | Concentration | | (AU) | control | (AU) | control | control |
| Non-irradiated control | | — | | 524783 | 95 | 1349 | 2 | 97 |
| Irradiated | Control | — | | 550661 | 100 | 81060 | 100 | 100 |
| conditions: | Ergothioneine | 0.5 | μM | 663197 | 120 | 45153 | 56 | 100 |
| UVA - | | 3 | μM | 606349 | 110 | 26636 | 33 | 96 |
| 4 J/cm² | | 30 | μM | 652512 | 118 | 20645 | 25 | 96 |

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications as fall within the scope of the appended claims.

All the features disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example of a generic series of equivalent or similar features.

### Other Embodiments

It is to be understood that while the invention has been described in conjunction with the detailed description thereof and accompanying figures, the foregoing description and accompanying figures are only intended to illustrate, and not limit the scope of the invention, which is defined by the scope of the appended claims. Other aspects, advantages, and modifications are within the scope of the following claims. All publications referenced herein are incorporated by reference in their entireties.

What is claimed is:

1. A topical composition for preventing or improving skin aging, comprising an active compound which is ergothioneine or an analog, derivative, or dermatologically acceptable salt thereof.

2. The composition of claim 1, wherein the active compound is present in an amount ranging from 0.01 to 20.0% by weight (w/w).

3. The composition of claim 1, wherein the active compound is present in an amount of about 10% by weight (w/w).

4. The composition of claim 1, wherein the active compound is present in an amount of about 0.5% by weight (w/w).

5. The composition of claim 1, wherein the analog or derivative is selected from the group consisting of S-Methyl-L-Ergothioneine, N-Desmethyl L-Ergothioneine Methyl Ester, N (Im)-Ethoxycarbonyl-S-ethoxycarbonyl L-Ergothioneine Methyl Ester lodide, N-Desmethyl L-Ergothioneine-D6 Methyl Ester, L-(+)-Ergothioneine-D3, ergothioneine sulfonate, and L-Ergothioneine Hydrochloride.

6. The composition of claim 1, wherein ergothioneine is L-ergothioneine.

7. The composition of claim 1, wherein the skin aging is photoaging.

8. The composition of claim 7, wherein the photoaging comprises one or more of wrinkling, scar tissue deposition, altered skin elasticity, altered skin color, altered skin texture, altered skin thickness, angioma, telangiectasia, sunburn, dryness, itchiness, neoplasia and precancerous growth.

9. The composition of claim 7, wherein preventing or improving photoaging is achieved by protection against UV-induced mtDNA deletion.

10. The composition of claim 1, wherein the composition comprises at least one additional agent, wherein the additional agent is selected from the group consisting of growth factor, hyaluronic acid, retinol/retinoic acid, hydrolyzed collagen or collagen peptides, vitamin C, vitamin E, cannabidiol, EGCG/Green tea, polyhydroxy acids, glycolic acid, aloe vera niacinamide, resveratrol, superoxide dismutase, tea tree oil, bakuchiol, grapeseed oil, petroleum jelly, and squalene.

11. The composition of claim 1, wherein the preventing or improving treating skin aging comprises one or more of the following effects: alleviating skin photoaging, diminishing skin wrinkles, improving skin elasticity, improving skin firmness, or improving skin's radiance, luminosity, or texture.

12. The composition of claim 11, wherein the composition improves skin's radiance, luminosity or texture.

13. The composition of claim 1, wherein the composition is in a form selected from the group consisting of a skin lotion, a skin softener, a skin toner, a skin cream, a sunscreen, an essence, a mask pack, a mask sheet, a soap, a shampoo, a cleaning foam, a cleaning lotion, a cleaning cream, an ointment, a gel, and a massage cream.

14. A method for preventing or improving skin aging in a subject in need thereof, comprising topically applying a composition of claim 1 to a skin area of the subject.

15. The method of claim 14, wherein the subject is human.

16. The method of claim 14, wherein the composition is present in an amount ranging from 0.01 to 20.0% by weight (w/w).

17. The method of claim 14, wherein the skin aging is photoaging.

18. The method of claim 17, wherein preventing or improving photoaging is achieved by protection against UV-induced mtDNA deletion.

19. The method of claim 14, wherein the preventing or improving treating skin aging comprises one or more of the following effects: alleviating skin photoaging, diminishing skin wrinkles, improving skin elasticity, improving skin firmness, or improving skin's radiance, luminosity, or texture.

20. Use of ergothioneine in manufacturing a composition capable of treating skin photoaging.
